(19) United States
(12) Patent Application Publication
(10) Pub. No.: US 2012/0289811 A1
(43) Pub. Date: Nov. 15, 2012

(54) MASK ON MONITOR HERNIA LOCATOR

(75) Inventors: Frank Viola, Sandy Hook, CT (US); Emily Davis, Roxbury, CT (US)

(73) Assignee: Tyco Healthcare Group LP

(21) Appl. No.: 13/437,197

(22) Filed: Apr. 2, 2012

Related U.S. Application Data
(60) Provisional application No. 61/485,661, filed on May 13, 2011.

(51) Int. Cl. A61B 6/00

(52) U.S. Cl. 600/407

(57) ABSTRACT

A method of tracking the location of a hernia defect is disclosed, the method including providing a display device, a camera adapted to transmit an image to the display device, a marking surface disposed on the display device and a marker adapted to alter the marking surface when applied to the marking surface. The method includes receiving an image of a hernia defect from the camera and displaying the received image on the display device and marking the shape and position of the hernia defect on the marking surface by applying the marker to the marking surface. Alternatively the display device may be marked by executing by the processor a computer program to mark the shape and position of the hernia defect on the display device.

### CROSS REFERENCE TO RELATED APPLICATION

The present application claims the benefit of and priority to U.S. Provisional Application Ser. No. 61/485,661, filed on May 13, 2011, the entire contents of which are incorporated herein by reference.

### BACKGROUND

1. Technical Field

The present disclosure relates generally to a method for tracking the location of a hernia defect. More particularly, the present disclosure relates a transparent mask placed on a monitor to locate a hernia defect and to track the location of the hernia defect during and after positioning and affixation of a hernia mesh to the hernia defect.

2. Description of Related Art

Hernias are abnormal protrusions of an organ or other body structure through a defect or natural opening in a covering membrane, e.g., a wall of a cavity that normally contains the organ or other body structure. For example, inguinal hernias are, typically, caused by soft tissue from the intestines protruding through the inguinal wall. Ventral hernias, on the other hand, are caused by internal organs pushing through to a weak spot in the abdominal wall.

The use of prosthetic mesh has now become accepted practice in the treatment of patients with both inguinal and ventral hernias, as well as other types of hernias, e.g., hiatal, femoral, umbilical, diaphragmatic, etc. To apply the mesh for hernia repair, a surgical region (i.e., adjacent the cavity wall) is, typically, insufflated. Subsequently, a surgeon selects points on the cavity wall where the surgeon believes a peripheral edge of the mesh, i.e., the expected corners of a mesh (assuming a rectangular mesh), will be affixed. In certain instances, prior to affixing the mesh, the mesh is, initially, held in position by pressing on the mesh from outside the body while observing the mesh through a laparoscope or, conversely, pressing upward against the mesh with the use of one or more suitable devices, e.g., an atraumatic grasper or the like. Thereafter, the surgical mesh is often affixed, i.e., sutured or fastened to the cavity wall by conventional suturing techniques, e.g., a needle with a suture (transfascial suture) is passed through the corners of the mesh and back through the tissue, or by conventional fastening techniques, e.g., the application of metal or polymer fasteners to the mesh and cavity wall. Unfortunately, once the mesh is deployed over the hernia, it obscures the position of the defect, hindering the surgeon from establishing an optimal mesh position prior to affixing the mesh.

Thus a method is needed for tracking the location of the hernia defect both before and after the mesh is positioned so that the surgeon can be assured of correct mesh placement.

### SUMMARY

A method of tracking the location of a hernia defect is disclosed, the method including providing a display device, an imaging device such as a camera adapted to transmit an image to the display device, a marking surface disposed on the display device and a marker adapted to alter the marking surface when applied to the marking surface. The method further includes receiving an image of a hernia defect from the camera and displaying the received image on the display device and marking the position of the hernia defect on the marking surface by applying the marker to the marking surface. Marking the position of the hernia defect may include marking a central position of the defect and also include marking the outline, shape, features and orientation of the defect.

The marking surface is a transparent member positionable on the display device and the method may further include positioning the transparent member on the display device where the transparent member may be removably attached to the display device. The transparent surface may be omitted and the marking be made directly on the screen. The marker may be adapted to leave a residue on the marking surface when applied to the marking surface and marking the shape and position of the hernia defect may include leaving the residue on the marking surface. The residue may also be removable from the marking surface after use.

Marking the shape and position of the hernia defect may include sketching an outline of the hernia defect with the marker.

The method may further include positioning a hernia mesh over the hernia defect and affixing the hernia mesh over the hernia defect where the position of the hernia mesh is adjusted relative to the hernia defect based on the shape and position marked on the marking surface by the marker before affixing the hernia mesh over the hernia defect.

The marking surface may alternatively be a touch sensitive screen where marking the shape and position of the hernia defect on the marking surface includes manipulating the touch sensitive screen to mark the shape and position of the hernia defect on the touch sensitive screen. The touch sensitive screen may be manipulated by a finger, a stylus, or any other method of manipulation known in the art. The marking may also be displayed as an overlay with the touch sensitive screen being manipulatable to transition the overlay between a first visible state and a second hidden state.

Another method of tracking the location of a hernia defect is disclosed, the method including providing a display device, a camera adapted to transmit an image to the display device, and a processor associated with the display device. The method further includes receiving an image of a hernia defect from the camera and displaying the received image on the display device and executing by the processor a computer program to mark the shape and position of the hernia defect on the display device. The processor executed computer program may highlight the hernia defect on the display device and may also provide a color contrast between the hernia defect and surrounding tissue on the display device.

The processor executed computer program may also track the location of the hernia defect upon movement of a patient's body and update the display device accordingly. The method further includes positioning a hernia mesh over the hernia defect and affixing the hernia mesh over the hernia defect and may adjust the position of the hernia mesh relative to the hernia defect based on the shape and position marked on the display device by the processor executed computer program before affixing the hernia mesh over the hernia defect. The marking of the shape and position of the hernia defect may also be displayed as an overlay where the display device is manipulatable to send an instruction to the processor to transition the overlay between a first visible state and a second hidden state.

### BRIEF DESCRIPTION OF THE DRAWINGS

The accompanying drawings, which are incorporated in and constitute a part of this specification, illustrate embodiments of the presently disclosed method, and together with a general description of the presently disclosed method given above, and the detailed description of the embodiments given below, serve to explain the principles of the presently disclosed method.

FIG. 1 is a view of the display device, transparent member, and camera in accordance with an embodiment of the present disclosure;

FIG. 2 is a view of the display device of an alternate embodiment of the present disclosure;

FIG. 3 is a flow diagram of the method of the embodiment of FIG. 1; and

FIG. 4 is a flow diagram of the method of the embodiment of FIG. 2.

### DETAILED DESCRIPTION

Disclosed herein is a method for tracking the location or position of a hernia defect.

Particular embodiments of the presently disclosed surgical device are described in detail with reference to the drawing figures wherein like reference numerals identify similar or identical elements. As used herein, the term “distal” refers to that portion which is farther from the user while the term “proximal” refers to that portion which is closer to the user or surgeon.

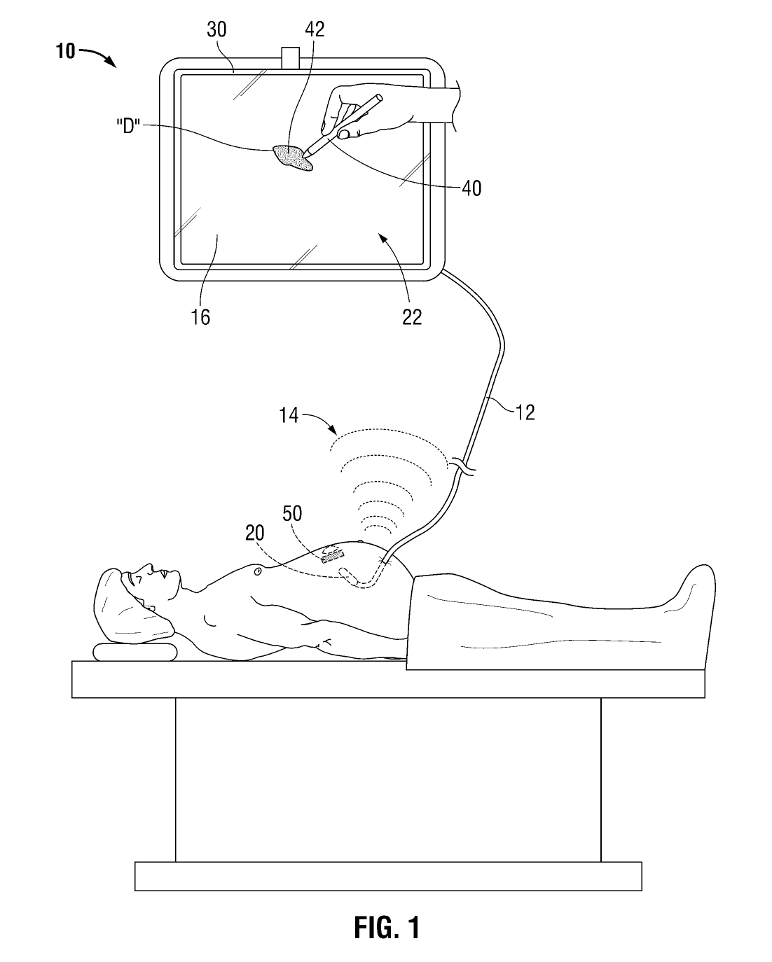

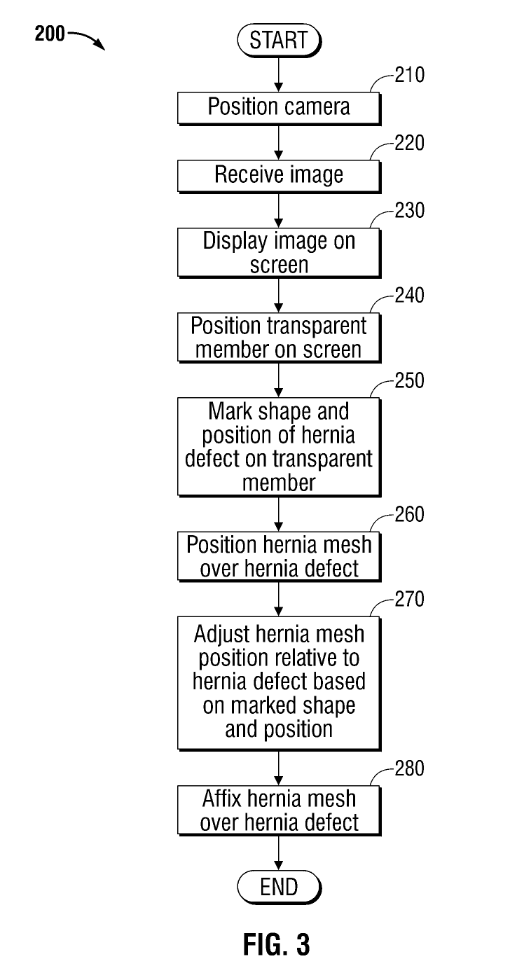

With reference to FIGS. 1 and 3, an embodiment is disclosed including a display device 10, a camera 20 adapted to capture an image 22 of a hernia defect “D” and to transmit image 22 of hernia defect “D” to display device 10, a transparent member 30 positionable on display device 10, and a marker 40 adapted to leave a residue 42 on transparent member 30 upon application thereto. Display device 10 includes a screen 16 for displaying image 22.

Camera 20 is positioned and fixedly mounted within the patient's body to capture image 22 of hernia defect “D” and to transmit the captured image 22 to display device 10 via a wire 12 or by a wireless connection 14. Camera 20 may capture image 22 as a live video feed.

Transparent member 30 is adapted to be positioned or applied to screen 16 and/or fixedly or removably attached to screen 16. Transparent member 30 may be attached or applied to screen 16 through any method known in the art including, for example, tape, adhesive, bonding, hook and loop (Velcro®), vinyl adhesion, etc. Transparent member 30 is made of a material which is receptive to residue 42 of marker 40 while still allowing a surgeon to see image 22 on screen 16. Transparent member 30 may be made from, for example, plastics, glass, or other transparent materials as known in the art. Transparent member 30 may be re-usable or cleanable where residue 42 of marker 40 may be “wiped off” or removed after use. In the instance when marker 40 may be “wiped off” or removed after use, the marking may optionally be made directly to the screen 16.

Marker 40 may, for example, be a grease pencil, a crayon, a pencil, a pen, chalk, marker, highlighter, or any other marker 40 known in the art which will leave residue 42 on transparent film 30 after being applied to transparent film 30. Residue 42 may be permanent or may instead be removable or erasable after use.

During use, a surgical site is prepared by inserting an introducer device through an incision or natural orifice in tissue and dissecting/insufflating a peritoneal space as disclosed in U.S. Pat. No. 6,679,900 to Kieturakis et al., the entirety of which is incorporated by reference herein. Camera 20 is then positioned and fixedly mounted in the dissected peritoneal space within a patient's body and oriented toward hernia defect “D” as desired. Camera 20 then transmits image 22 to display 10 for output to screen 16. Screen 16 displays image 22 of hernia defect “D” for a surgeon to examine. Once the surgeon locates hernia defect “D” the surgeon positions or attaches transparent member 30 to screen 16 and marks transparent member 30 with marker 40. The surgeon marks transparent member 30 in a location corresponding to the position of hernia defect “D” on screen 16 and may also outline the shape and position of hernia defect “D” on transparent member 30. Once hernia defect “D” is sufficiently marked on transparent member 30, the surgeon may insert and position a hernia mesh 50 over hernia defect “D” through the use of an introducer device (not shown), as seen in U.S. Pat. No. 6,679,900 discussed above. Although positioning hernia mesh 50 over hernia defect “D” obstructs camera 20 from viewing hernia defect “D” such that image 22 no longer shows hernia defect “D”, the surgeon is still able to track the position and shape of hernia defect “D” due to the shape and position of residue 42 on transparent member 30. This provides the surgeon with additional assurance that hernia mesh 50 is being positioned properly over hernia defect “D”. The surgeon now affixes hernia mesh 50 to hernia defect “D” to complete the operation.

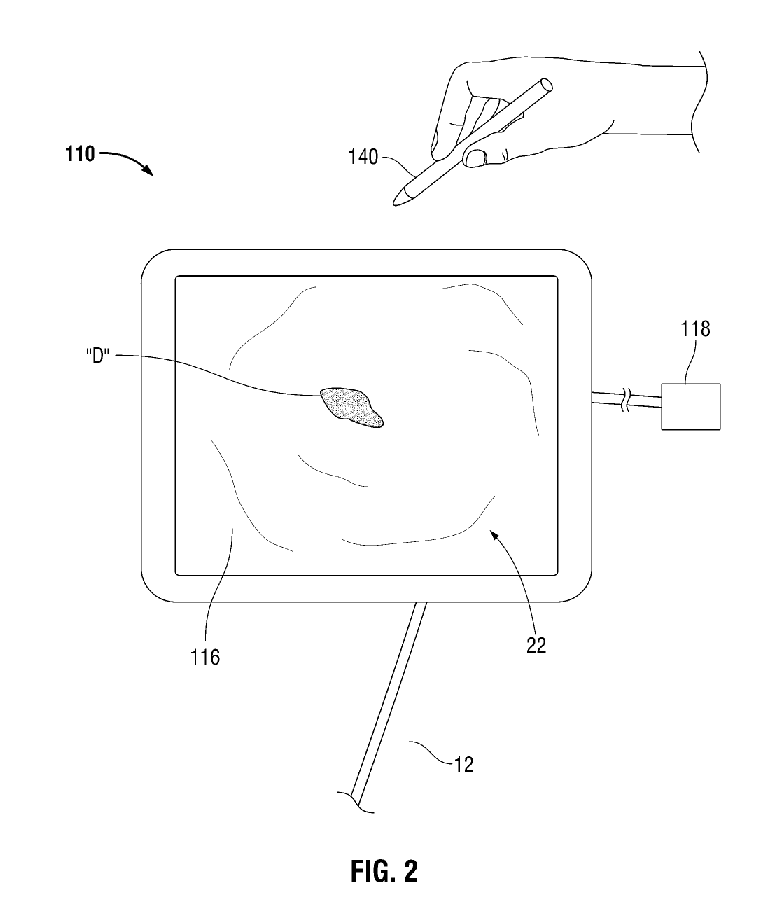

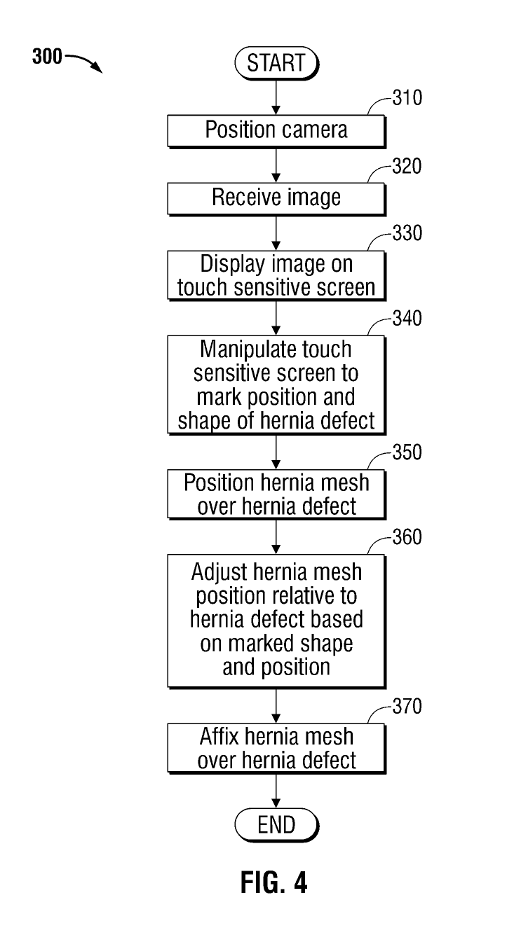

In another embodiment, with reference to FIGS. 2 and 4, display device 110 includes a screen 116 which is touch sensitive, and a processor 118 disposed in communication with display device 110. Touch sensitive screen 116 provides a function similar to that of transparent member 30 where touch sensitive screen 116 may be manipulated to mark or outline a hernia defect “D” shown in an image 22 displayed thereon. Touch sensitive screen 116 may be manipulated by a finger, or may be manipulated by another marker 140 such as a stylus, a computer mouse, or any other method known in the art. Alternatively hernia defect “D” may be marked or outlined automatically by a computer program executed by processor 118. In addition to being marked or outlined, hernia defect “D” may also be highlighted or color contrasted with respect to surrounding tissue through the use of the computer program executed by processor 118. The computer program executed by processor 118 may also track movement of hernia defect “D” due to motion of the body including, for example, insufflation and breathing. The computer program executed on processor 118 may also be utilized in cases where display device 110 does not include a touch sensitive screen 116. The marking or outline of hernia defect “D” may also be stored on a separate layer or overlay such that upon manipulation of touch sensitive screen 116 or display 110 the separate layer or overlay may be transitioned between a visible state and a hidden state. The separate layer or overlay may also be transitioned between the visible state and the hidden state by sending an instruction to processor 118.

During use, the surgical site is prepared as discussed above and disclosed in U.S. Pat. No. 6,679,900 and camera 20 is positioned and fixedly mounted within a patient's body and oriented toward hernia defect “D” as desired. Camera 20 then transmits image 22 to display 110 for output to touch sensitive screen 116. Touch sensitive screen 116 displays image 22 of hernia defect “D” for a surgeon to examine. Once the surgeon locates hernia defect “D” the surgeon manipulates touch sensitive screen 116 with a finger, stylus or other marker 140 to mark or outline hernia defect “D”. Alternatively, the computer program executed on processor 118 may automatically mark or outline hernia defect “D” or may mark or outline hernia defect “D” upon input of a command by the surgeon. Once hernia defect “D” is sufficiently marked on touch sensitive screen 116, the surgeon may insert and position hernia mesh 50 over hernia defect “D” as discussed above with respect to U.S. Pat. No. 6,679,900. Although positioning hernia mesh 50 over hernia defect “D” obstructs camera 20 from viewing hernia defect “D” such that image 22 no longer shows hernia defect “D”, the surgeon is still able to track the position and shape of hernia defect “D” based on the shape and outline provided on touch sensitive screen 116. The surgeon is further able to track the position and shape of hernia defect “D” during motion of the patient through the use of the computer program executed by processor 118. The computer program tracks the position of hernia defect “D” and updates display device 110 with the current position of hernia defect “D” even though image 22 is obscured by hernia mesh 50. This provides the surgeon with additional assurance that hernia mesh 50 is being positioned properly over hernia defect “D”. The surgeon now affixes hernia mesh 50 to hernia defect “D” to complete the operation as described above.

FIG. 3 is a flow diagram of a process 200 for tracking the location of a hernia defect “D” using transparent member 30. Prior to process 200, a surgical instrument or object is inserted into an incision or natural orifice to prepare the peritoneal space for the operation by dissecting and insufflating the peritoneal space as disclosed in U.S. Pat. No. 6,679,900. Process 200 starts at step 210 with a surgeon positioning camera 20 within a patient's body, in the peritoneal space, and orienting camera 20 toward a hernia defect “D”. Next at step 220, display device 10 receives image 22 from camera 20. Next at step 230, display device 10 displays received image 22 on screen 16 for the surgeon to examine. Next at step 240, the surgeon positions transparent member 30 on or attaches transparent member 30 to screen 16 as described above through the use of adhesive, staples, hook and loop, or other methods as known in the art. Next at step 250, after the surgeon locates the hernia defect “D” the surgeon marks the shape and position of the hernia defect “D” on transparent member 30 as described above using marker 40. Next at step 260, after the shape and position of hernia defect “D” is marked on transparent member 30 the surgeon positions hernia mesh 50 over the hernia defect “D”. Next at step 270, the surgeon adjusts the position of hernia mesh 50 relative to hernia defect “D” based on the marked shape and position. Even though hernia mesh 50 now obscures image 22 received from camera 20, the surgeon is still able to track the location of hernia defect “D” due to the markings on transparent member 30. Finally at step 280, once the surgeon is assured of the correct placement of mesh 50, the surgeon affixes mesh 50 in position as seen in U.S. Pat. No. 6,679,900, discussed above.

FIG. 4. is a flow diagram of an alternate process 300 for tracking the location of hernia defect “D”. As with process 200 above, process 300 begins after the peritoneal space is prepared. Process 300 starts at step 310 with a surgeon positioning camera 20 within a patient's body, in the peritoneal space, and orienting camera 20 toward a hernia defect “D”. Next at step 320, display device 110 receives image 22 from camera 20. Next at step 330, display device 110 displays received image 22 on touch sensitive screen 116 for the surgeon to examine. Next at step 340, the surgeon manipulates touch sensitive screen 116 as described above to mark the position and shape of hernia defect “D” on touch sensitive screen 116. Step 340 may also include the use of the computer program executed by processor 118 to further enhance the marking of the position and shape of hernia defect “D” and in addition to adjust the position and shape of hernia defect “D” on touch sensitive screen 116 after movement of the patient. Next at step 350, the surgeon positions hernia mesh 50 over hernia defect “D”. Next at step 360, the surgeon adjusts hernia mesh 50 relative to hernia defect “D” based on the shape and position marked on touch sensitive screen 116. Even though hernia mesh 50 now obscures image 22 received from camera 20, the surgeon is still able to track the location of hernia defect “D” due to the markings on the touch screen and/or the output from the computer program executed on processor 118. Finally at step 370, once the surgeon is assured of the correct placement of mesh 50, the surgeon affixes mesh 50 in position as described in U.S. Pat. No. 6,679,900, discussed above.

Although the present disclosure has been described with respect to preferred embodiments, it will be readily apparent, to those having ordinary skill in the art that changes and modifications may be made thereto without departing from the spirit or scope of the subject method.

What is claimed is:

1. A method of tracking the location of a hernia defect comprising:
providing a display device;
providing a imaging device, the imaging device adapted to transmit an image to the display device;
providing a marking surface disposed on the display device;
providing a marker, the marker adapted to alter the marking surface when applied to the marking surface;
receiving an image of a hernia defect from the imaging device and displaying the received image on the display device; and
marking the position of the hernia defect on the marking surface by applying the marker to the marking surface.

2. The method of claim 1, wherein the marking surface is a transparent member positionable on the display device, the method further comprising the step of positioning the transparent member on the display device.

3. The method of claim 2, wherein positioning the transparent member on the display device comprises removably attaching the transparent member to the display device.

4. The method of claim 1, wherein the marker is adapted to leave residue on the marking surface when applied to the marking surface, the step of marking the position of the hernia defect including leaving a residue on the marking surface.

5. The method of claim 4, wherein the residue is removable from the marking surface after use.

6. The method of claim 1, wherein marking the position of the hernia defect comprises marking the shape of the hernia defect with the marker.

7. The method of claim 6, wherein marking the shape of the hernia defect comprises sketching an outline of the hernia defect with the marker.

8. The method of claim 1, further comprising positioning a hernia mesh over the hernia defect and affixing the hernia mesh over the hernia defect.

9. The method of claim 1, further comprising the step of adjusting the position of the hernia mesh relative to the hernia defect based on the position marked on the marking surface by the marker before affixing the hernia mesh over the hernia defect.

10. The method of claim 1, wherein the marking surface is a touch sensitive screen.

11. The method of claim 10, wherein the step of marking the position of the hernia defect on the marking surface comprises manipulating the touch sensitive screen to mark the position of the hernia defect on the touch sensitive screen.

12. The method of claim 1 wherein the step of marking the position of the hernia defect on the marking surface comprises using a light beam.

13. The method of claim 10, wherein the marking is displayed as an overlay, the touch sensitive screen capable of sending an instruction to transition the overlay between a first visible state and a second hidden state.

14. A method of tracking the location of a hernia defect comprising:
providing a display device;
providing a imaging device, the imaging device adapted to transmit an image to the display device;
providing a processor associated with the display device;
receiving an image of a hernia defect from the imaging device and displaying the received image on the display device; and
executing by the processor a computer program to mark the position of the hernia defect on the display device.

15. The method of claim 14, wherein the processor executed computer program highlights the hernia defect on the display device.

16. The method of claim 14, wherein the processor executed computer program provides a color contrast between the hernia defect and surrounding tissue on the display device.

17. The method of claim 14, wherein the processor executed computer program tracks the location of the hernia defect upon movement of a patient's body and updates the display device accordingly.

18. The method of claim 14, further comprising positioning a hernia mesh over the hernia defect and affixing the hernia mesh over the hernia defect.

19. The method of claim 18, further comprising the step of adjusting the position of the hernia mesh relative to the hernia defect based on position marked on the display device by the processor executed computer program before affixing the hernia mesh over the hernia defect.

20. The method of claim 14, wherein the marking of position of the hernia defect is displayed as an overlay, the display device capable of sending an instruction to the processor to transition the overlay between a first visible state and a second hidden state.
